(19) United States
(12) Patent Application Publication
(10) Pub. No.: US 2014/0277569 A1
(43) Pub. Date: Sep. 18, 2014

(54) HYBRID OSTEOINDUCTIVE BONE GRAFT

(71) Applicant: WARSAW ORTHOPEDIC, INC., Warsaw, IN (US)

(72) Inventor: ERIC C. LANGE, Collierville, TN (US)

(73) Assignee: Warsaw Orthopedic, Inc., Warsaw, IN (US)

(21) Appl. No.: 13/800,977

(22) Filed: Mar. 13, 2013

(51) Int. Cl. A61F 2/28

(52) U.S. Cl. CPC A61F 2/28
USPC 623/23.51

(57) ABSTRACT

A bone implant includes a first surface and a second surface. The first and second surfaces include a bioresorbable material. A third surface includes a biocompatible material disposed between the first and second surfaces. The third surface extends between a first end and a second end. The first and second ends each include an inner surface defining a cavity configured for disposal of a spinous process. The bioresorbable material of the first and second surfaces is a faster resorbing material than the biocompatible material of the third surface. The third surface provides structural integrity of the implant to maintain distraction between spinous processes so that the first and second surfaces fuse with at least a portion of the spine.

### TECHNICAL FIELD

The present disclosure relates generally to instruments and devices for treating musculoskeletal disorders. In particular, the present disclosure relates to structural osteoinductive bone grafts for treating musculoskeletal disorders.

### BACKGROUND

The rapid and effective repair of bone defects caused by injury, disease, wounds, or surgery is a goal of orthopedic surgery. Toward this end, a number of bone implants have been used or proposed for use in the repair of bone defects. The biological, physical, and mechanical properties of the bone implants are among the major factors influencing their suitability and performance in various orthopedic applications.

Bone implants are used to repair bone that has been damaged by disease, trauma, or surgery. Bone implants may be utilized when healing is impaired in the presence of certain drugs or in disease states such as diabetes, when a large amount of bone or disc material is removed during surgery, or when bone fusion is needed to create stability. In some types of spinal fusion, for example, bone implants are used to replace the cushioning disc material between the vertebrae or to repair a degenerative facet joint.

One type of bone implant is the bone graft. Typically, bone graft (e.g., osteograft) materials may include both synthetic and natural bone. Natural bone may be taken from the graft recipient (autograft) or may be taken from another source (allograft), such as a cadaver, or (xenograft), such as bovine. Autografts have advantages such as decreased immunogenicity and greater osteoinductive potential, but there can also be problems with donor site morbidity and a limited supply of suitable bone for grafting. On the other hand, allografts are available in greater supply and can be stored for years. However, allografts tend to be less osteoinductive.

Osteoconduction and osteoinduction both contribute to bone formation. A graft material is osteoconductive if it provides a structural framework or microscopic and macroscopic scaffolding for cells and cellular materials that are involved in bone formation (e.g., osteoclasts, osteoblasts, vasculature, mesenchymal cells).

Osteoinductive material, on the other hand, stimulates differentiation of host mesenchymal cells into chondroblasts and osteoblasts. Natural bone allograft materials can comprise either cortical or cancellous bone. A distinguishing feature of cancellous bone is its high level of porosity relative to that of cortical bone, providing more free surfaces and more of the cellular constituents that are retained on these surfaces. It provides both an osteoinductive and osteoconductive graft material, but generally does not have significant load-bearing capacity. Optimal enhancement of bone formation is generally thought to require a minimum threshold quantity of cancellous bone, however. Cortical (compact) bone has greater strength or load-bearing capacity than cancellous bone, but is less osteoconductive. In humans for example, only approximately twenty percent of large cortical allografts are completely incorporated at five years. Delayed or incomplete incorporation may allow micromotion, leading to host bone resorption around the allograft. A more optimal bone graft material would combine significant load-bearing capacity with both osteoinductive and osteoconductive properties, and much effort has been directed toward developing such a graft material.

Some allografts comprise mammalian cadaver bone treated to remove all soft tissue, including marrow and blood, and then textured to form a multiplicity of holes of selected size, spacing, and depth. The textured bone section is then immersed and demineralized, for example, in a dilute acid bath. Demineralizing the bone exposes osteoinductive factors, but extensive demineralization of bone also decreases its mechanical strength.

Allografts have also been formed of organic bone matrix with perforations that extend from one surface, through the matrix, to the other surface to provide continuous channels between opposite surfaces. The organic bone matrix is produced by partial or complete demineralization of natural bone. Although the perforations increase the scaffolding potential of the graft material and may be filled with osteoinductive material as well, perforating organic bone matrix through the entire diameter of the graft decreases its load-bearing capacity.

Partially-demineralized cortical bone constructs may be surface-demineralized to prepare the graft to be soaked in bone growth-promoting substances such as bone morphogenetic protein (BMP). Although this design allows greater exposure of the surrounding tissue to growth-promoting factors, the surface demineralization necessary to adhere a substantial amount of growth-promoting factors to the graft material decreases the allograft's mechanical strength.

What is needed is a bone implant that combines the osteoinductive and osteoconductive properties of cancellous bone with the load-bearing capacity provided by cortical allograft materials. Compositions and methods are needed that facilitate bone remodeling and new bone growth, and integration of the bone implant (e.g., allograft) into host bone.

### SUMMARY

In one embodiment, in accordance with the principles of the present disclosure, a bone implant is provided. The bone implant includes a first surface and a second surface. The first and second surfaces include a bioresorbable material. A third surface includes a biocompatible material disposed between the first and second surfaces. The third surface extends between a first end and a second end. The first and second ends each include an inner surface defining a cavity configured for disposal of a spinous process. The bioresorbable material of the first and second surfaces is a faster resorbing material than the biocompatible material of the third surface. The third surface provides structural integrity of the implant to maintain distraction between spinous processes so that the first and second surfaces fuse with at least a portion of the spine.

In one embodiment, in accordance with the principles of the present disclosure, a bone implant is provided. The bone implant includes a first layer including an upper surface and a lower surface. The first layer includes a bioresorbable material. A second layer includes a biocompatible material attached to the lower surface of the first layer. The second layer extends between a first end and a second end. The first and second ends each include an inner surface defining a cavity configured for disposal of a spinous process. The bioresorbable material of the first layer is a faster resorbing material than the biocompatible material of the second layer. The second layer provides structural integrity of the implant to maintain distraction between spinous processes so that the first layer fuses with at least a portion of the spine.

In one embodiment, in accordance with the principles of the present disclosure, a bone implant is provided. The bone implant includes a first bioresorbable polymer mesh bag and a second bioresorbable polymer mesh bag. The first and second mesh bags each include demineralized bone chips disposed therein. The bone implant further includes a surface. The surface includes cortical bone. The surface is disposed between and connected to the first and second mesh bags. The surface extends between a first end and a second end. The first and second ends each include an inner surface defining a cavity configured for disposal of a spinous process. The surface provides structural integrity of the implant to maintain distraction between spinous processes so that the demineralized bone chips fuse with at least a portion of the spine.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE DRAWINGS

The present disclosure will become more readily apparent from the specific description accompanied by the following drawings, in which:

FIG. 1 is a perspective view of components of one embodiment of a system in accordance with the principles of the present disclosure;

FIG. 2 is a side view of the components shown in FIG. 1;

FIG. 3 is a plan view of components of one embodiment of a system in accordance with the principles of the present disclosure;

FIG. 4 is a perspective view of components of one embodiment of a system in accordance with the principles of the present disclosure;

FIG. 5 is a side view of the components shown in FIG. 4; and

FIG. 6 is a perspective view of the components shown in FIG. 1 disposed with vertebrae.

It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present application. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical are as precise as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

Additionally, unless defined otherwise or apparent from context, all technical and scientific terms used herein have the same meanings as commonly understood by one of ordinary skill in the art to which this invention belongs.

Unless explicitly stated or apparent from context, the following terms are phrases have the definitions provided below:

### DEFINITIONS

It is noted that, as used in this specification and the appended claims, the singular forms “a,” “an,” and “the,” include plural referents unless expressly and unequivocally limited to one referent. Thus, for example, reference to “an allograft” includes one, two, three or more allografts.

The term “biodegradable” includes that all or parts of the carrier and/or implant will degrade over time by the action of enzymes, by hydrolytic action and/or by other similar mechanisms in the human body. In various embodiments, “biodegradable” includes that the carrier and/or implant can break down or degrade within the body to non-toxic components after or while a therapeutic agent has been or is being released. By “bioerodible” it is meant that the carrier and/or implant will erode or degrade over time due, at least in part, to contact with substances found in the surrounding tissue, fluids or by cellular action. By “bioabsorbable” or “bioresorbable” it is meant that the carrier and/or implant will be broken down and absorbed within the human body, for example, by a cell or tissue. “Biocompatible” means that the allograft will not cause substantial tissue irritation or necrosis at the target tissue site.

The term “mammal” refers to organisms from the taxonomy class “mammalian,” including but not limited to humans, other primates such as chimpanzees, apes, orangutans and monkeys, rats, mice, cats, dogs, cows, horses, etc.

“A “therapeutically effective amount” or “effective amount” is such that when administered, the drug (e.g., growth factor) results in alteration of the biological activity, such as, for example, promotion of bone, cartilage and/or other tissue (e.g., vascular tissue) growth, inhibition of inflammation, reduction or alleviation of pain, improvement in the condition through inhibition of an immunologic response, etc. The dosage administered to a patient can be as single or multiple doses depending upon a variety of factors, including the drug's administered pharmacokinetic properties, the route of administration, patient conditions and characteristics (sex, age, body weight, health, size, etc.), extent of symptoms, concurrent treatments, frequency of treatment and the effect desired. In some embodiments the implant is designed for immediate release. In other embodiments the implant is designed for sustained release. In other embodiments, the implant comprises one or more immediate release surfaces and one or more sustained release surfaces.

The phrase “immediate release” is used herein to refer to one or more therapeutic agent(s) that is introduced into the body and that is allowed to dissolve in or become absorbed at the location to which it is administered, with no intention of delaying or prolonging the dissolution or absorption of the drug.

The phrases “sustained release” and “sustain release” (also referred to as extended release or controlled release) are used herein to refer to one or more therapeutic agent(s) that is introduced into the body of a human or other mammal and continuously or continually releases a stream of one or more therapeutic agents over a predetermined time period and at a therapeutic level sufficient to achieve a desired therapeutic effect throughout the predetermined time period.

The terms “treating” and “treatment” when used in connection with a disease or condition refer to executing a protocol that may include a bone repair procedure, where the bone implant and/or one or more drugs are administered to a patient (human, other normal or otherwise or other mammal), in an effort to alleviate signs or symptoms of the disease or condition or immunological response. Alleviation can occur prior to signs or symptoms of the disease or condition appearing, as well as after their appearance. Thus, treating or treatment includes preventing or prevention of disease or undesirable condition. In addition, treating, treatment, preventing or prevention do not require complete alleviation of signs or symptoms, does not require a cure, and specifically includes protocols that have only a marginal effect on the patient.

The term “bone,” as used herein, refers to bone that is cortical, cancellous or cortico-cancellous of autogenous, allogenic, xenogenic, or transgenic origin.

The term “allograft” refers to a graft of tissue obtained from a donor of the same species as, but with a different genetic make-up from, the recipient, as a tissue transplant between two humans.

The term “autologous” refers to being derived or transferred from the same individual's body, such as for example an autologous bone marrow transplant.

The term “osteoconductive,” as used herein, refers to the ability of a non-osteoinductive substance to serve as a suitable template or substance along which bone may grow.

The term “osteoinductive,” as used herein, refers to the quality of being able to recruit cells from the host that have the potential to stimulate new bone formation. Any material that can induce the formation of ectopic bone in the soft tissue of an animal is considered osteoinductive.

The term “osteoinduction” refers to the ability to stimulate the proliferation and differentiation of pluripotent mesenchymal stem cells (MSCs). In endochondral bone formation, stem cells differentiate into chondroblasts and chondrocytes, laying down a cartilaginous ECM, which subsequently calcifies and is remodeled into lamellar bone. In intramembranous bone formation, the stem cells differentiate directly into osteoblasts, which form bone through direct mechanisms. Osteoinduction can be stimulated by osteogenic growth factors, although some ECM proteins can also drive progenitor cells toward the osteogenic phenotype.

The term “osteoconduction” refers to the ability to stimulate the attachment, migration, and distribution of vascular and osteogenic cells within the graft material. The physical characteristics that affect the graft's osteoconductive activity include porosity, pore size, and three-dimensional architecture. In addition, direct biochemical interactions between matrix proteins and cell surface receptors play a major role in the host's response to the graft material.

The term “osteogenic” refers to the ability of a graft material to produce bone independently. To have direct osteogenic activity, the graft must contain cellular components that directly induce bone formation. For example, an allograft seeded with activated MSCs would have the potential to induce bone formation directly, without recruitment and activation of host MSC populations. Because many osteoconductive allografts also have the ability to bind and deliver bioactive molecules, their osteoinductive potential will be greatly enhanced.

The term “osteoimplant,” as used herein, refers to any bone-derived implant prepared in accordance with the embodiments of this disclosure and therefore is intended to include expressions such as bone membrane, bone graft, etc.

The term “patient” refers to a biological system to which a treatment can be administered. A biological system can include, for example, an individual cell, a set of cells (e.g., a cell culture), an organ, or a tissue. Additionally, the term “patient” can refer to animals, including, without limitation, humans.

The term “xenograft” refers to tissue or organs from an individual of one species transplanted into or grafted onto an organism of another species, genus, or family.

The term “demineralized,” as used herein, refers to any material generated by removing mineral material from tissue, e.g., bone tissue. In certain embodiments, the demineralized compositions described herein include preparations containing less than 5% calcium and preferably less than 1% calcium by weight. Partially demineralized bone (e.g., preparations with greater than 5% calcium by weight but containing less than 100% of the original starting amount of calcium) is also considered within the scope of the disclosure. In some embodiments, demineralized bone has less than 95% of its original mineral content. Demineralized is intended to encompass such expressions as “substantially demineralized,” “partially demineralized,” and “fully demineralized.” In some embodiments, part or all of the surface of the bone can be demineralized. For example, part or all of the surface of the allograft can be demineralized to a depth of from about 100 to about 5000 microns, or about 150 microns to about 1000 microns. If desired, the outer surface of the intervertebral implant can be masked with an acid resistant coating or otherwise treated to selectively demineralize unmasked portions of the outer surface of the intervertebral implant so that the surface demineralization is at discrete positions on the implant.

The term “demineralized bone matrix,” as used herein, refers to any material generated by removing mineral material from bone tissue. In some embodiments, the DBM compositions as used herein include preparations containing less than 5% calcium and preferably less than 1% calcium by weight. Partially demineralized bone (e.g., preparations with greater than 5% calcium by weight but containing less than 100% of the original starting amount of calcium) are also considered within the scope of the disclosure.

The term “superficially demineralized,” as used herein, refers to bone-derived elements possessing at least about 90 weight percent of their original inorganic mineral content, the expression “partially demineralized” as used herein refers to bone-derived elements possessing from about 8 to about 90 weight percent of their original inorganic mineral content and the expression “fully demineralized” as used herein refers to bone containing less than 8% of its original mineral context.

The terms “pulverized bone”, “powdered bone” or “bone powder” as used herein, refers to bone particles of a wide range of average particle size ranging from relatively fine powders to coarse grains and even larger chips.

Demineralized bone matrix comprises bone fibers, chips, powder and/or shards. Fibers include bone elements whose average length to average thickness ratio or aspect ratio of the fiber is from about 50:1 to about 1000:1. In overall appearance the fibrous bone elements can be described as elongated bone fibers, threads, narrow strips, or thin sheets. Often, where thin sheets are produced, their edges tend to curl up toward each other. The fibrous bone elements can be substantially linear in appearance or they can be coiled to resemble springs. In some embodiments, the elongated bone fibers are of irregular shapes including, for example, linear, serpentine or curved shapes. The elongated bone fibers are preferably demineralized however some of the original mineral content may be retained when desirable for a particular embodiment.

Non-fibrous, as used herein, refers to elements that have an average width substantially larger than the average thickness of the fibrous bone element or aspect ratio of less than from about 50:1 to about 1000:1. In some embodiments, the non-fibrous bone elements are shaped in a substantially regular manner or specific configuration, for example, triangular prism, sphere, cube, cylinder and other regular shapes. By contrast, particles such as chips, shards, or powders possess irregular or random geometries. It should be understood that some variation in dimension will occur in the production of the elements of this application and elements demonstrating such variability in dimension are within the scope of this application and are intended to be understood herein as being within the boundaries established by the expressions “mostly irregular” and “mostly regular”.

Reference will now be made in detail to certain embodiments of the invention. While the invention will be described in conjunction with the illustrated embodiments, it will be understood that they are not intended to limit the invention to those embodiments. On the contrary, the disclosure is intended to cover all alternatives, modifications, and equivalents that may be included within the invention as defined by the appended claims.

Compositions are provided that facilitate bone remodeling and new bone growth, and integration of the bone implant (e.g., allograft) into host bone. In one embodiment, a structural bone graft is provided that is capable of maintaining distraction between the spinous processes and also incorporates an osteoinductive portion with a much higher propensity to fuse with the underlying host bone. In one embodiment, the bone implant includes a structural, cortical bone center portion combined with two osteoinductive portions disposed adjacent the cortical bone center portion. The osteoinductive portions of the hybrid bone graft may be manufactured utilizing various configurations of demineralized bone.

Current structural allograft implants can be made from dense cortical bone requiring significant time for the host bone to remodel the allograft interface surface via osteoclastic resorption and eventual deposition of new bone into the allograft. By employing the bone implant of the current application that includes demineralized bone matrix, such as, for example, demineralized bone chips, fibers and/or powders relatively loosely packed within a bioresorbable polymer mesh bag, attached to the dense cortical bone center portion, the fusion process can be accelerated while simultaneously maintaining the distraction of the spinous processes.

In some embodiments, the portion of the allograft that is not demineralized, such as, for example, the cortical bone center portion, comprises load bearing and/or higher compressive strength allograft material. In some embodiments, the portion of the allograft that is not load bearing comprises demineralized bone material that also has a low compressive strength.

In some embodiments, the implant device contacts host bone and the implant device comprises from about 1% to about 30% or from about 5% to about 25% by weight of demineralized bone material.

In some embodiments, the bone allograft material comprises demineralized bone matrix fibers and demineralized bone matrix powder in a ratio of 25:75 to about 75:25 fibers to chips.

The healing process also exposes some of the inherent bone growth factors in the cortical allograft material to further facilitate remodeling and new bone formation.

Demineralized bone matrix (DBM) is demineralized allograft bone with osteoinductive activity. DBM is prepared by acid extraction of allograft bone, resulting in loss of most of the mineralized component but retention of collagen and noncollagenous proteins, including growth factors. DBM does not contain osteoprogenitor cells, but the efficacy of a demineralized bone matrix as a bone-graft substitute or extender may be influenced by a number of factors, including the sterilization process, the carrier, the total amount of bone morphogenetic protein (BMP) present, and the ratios of the different BMPs present. DBM includes demineralized pieces of cortical bone to expose the osteoinductive proteins contained in the matrix. DBM is mostly an osteoinductive product, but lacks enough induction to be used on its own in challenging healing environments such as posterolateral spine fusion.

In one embodiment, DBM powder can range in average particle size from about 0.0001 to about 1.2 cm and from about 0.002 to about 1 cm. The bone powder can be obtained from cortical, cancellous and/or corticocancellous allogenic or xenogenic bone tissue. In general, allogenic bone tissue is preferred as the source of the bone powder.

According to some embodiments of the disclosure, the demineralized bone matrix portions of the bone implant may comprise demineralized bone matrix fibers and/or demineralized bone matrix chips. In some embodiments, the demineralized bone matrix may comprise demineralized bone matrix fibers and demineralized bone matrix chips in a 30:60 ratio. The bone graft materials of the present disclosure include those structures that have been modified in such a way that the original chemical forces naturally present have been altered to attract and bind molecules, including, without limitation, growth factors and/or cells, including cultured cells.

Namely, the demineralized allograft bone material may be further modified such that the original chemical forces naturally present have been altered to attract and bind growth factors, other proteins and cells affecting osteogenesis, osteoconduction and osteoinduction. For example, a the demineralized bone matrix portions of the bone implant may be modified to provide an ionic gradient to produce a modified demineralized bone matrix portion, such that implanting the modified demineralized bone matrix portion results in enhanced ingrowth of host bone.

In one embodiment, an ionic force change agent may be applied to modify the demineralized bone matrix portions. The demineralized bone matrix portions may comprise, e.g., a demineralized bone matrix (DBM) comprising fibers, particles and any combination of thereof disposed within a bioresorbable polymer mesh bag.

The ionic force change agent may be applied to the entire demineralized allograft bone material or to selected portions/surfaces thereof.

The ionic force change agent may be a binding agent, which modifies the faster resorbing demineralized bone matrix portions to bind molecules, such as, for example, DBM, growth factors, or cells, such as, for example, cultured cells, or a combination of molecules and cells. In the practice of the disclosure the growth factors include but are not limited to BMP-2, rhBMP-2, BMP-4, rhBMP-4, BMP-6, rhBMP-6, BMP-7(OP-1), rhBMP-7, GDF-5, LIM mineralization protein, platelet derived growth factor (PDGF), transforming growth factor-β (TGF-β), insulin-related growth factor-I (IGF-I), insulin-related growth factor-II (IGF-II), fibroblast growth factor (FGF), beta-2-microglobulin (BDGF II), and rhGDF-5. A person of ordinary skill in the art will appreciate that the disclosure is not limited to growth factors only. Other molecules can also be employed in the disclosure. For example, tartrate-resistant acid phosphatase, which is not a growth factor, may also be used in the disclosure.

An adhesive may be applied to the DBM chips, powders and/or fibers. The adhesive material may comprise polymers having hydroxyl, carboxyl, and/or amine groups. In some embodiments, polymers having hydroxyl groups include synthetic polysaccharides, such as for example, cellulose derivatives, such as cellulose ethers (e.g., hydroxypropylcellulose). In some embodiments, the synthetic polymers having a carboxyl group, may comprise poly(acrylic acid), poly(methacrylic acid), poly(vinyl pyrrolidone acrylic acid-N-hydroxysuccinimide), and poly(vinyl pyrrolidone-acrylic acid-acrylic acid-N-hydroxysuccinimide) terpolymer. For example, poly(acrylic acid) with a molecular weight greater than 250,000 or 500,000 may exhibit particularly good adhesive performance. In some embodiments, the adhesive can be a polymer having a molecular weight of about 2,000 to about 5,000, or about 10,000 to about 20,000 or about 30,000 to about 40,000.

In some embodiments, the adhesive can comprise imido ester, p-nitrophenyl carbonate, N-hydroxysuccinimide ester, epoxide, isocyanate, acrylate, vinyl sulfone, orthopyridyl-disulfide, maleimide, aldehyde, iodoacetamide or a combination thereof. In some embodiments, the adhesive material can comprise at least one of fibrin, a cyanoacrylate (e.g., N-butyl-2-cyanoacrylate, 2-octyl-cyanoacrylate, etc.), a collagen-based component, a glutaraldehyde glue, a hydrogel, gelatin, an albumin solder, and/or a chitosan adhesives. In some embodiments, the hydrogel comprises acetoacetate esters crosslinked with amino groups or polyethers as mentioned in U.S. Pat. No. 4,708,821. In some embodiments, the adhesive material can comprise poly(hydroxylic) compounds derivatized with acetoacetate groups and/or polyamino compounds derivatized with acetoacetamide groups by themselves or the combination of these compounds crosslinked with an amino-functional crosslinking compounds.

The adhesive can be a solvent based adhesive, a polymer dispersion adhesive, a contact adhesive, a pressure sensitive adhesive, a reactive adhesive, such as for example multi-part adhesives, one part adhesives, heat curing adhesives, moisture curing adhesives, or a combination thereof or the like. The adhesive can be natural or synthetic or a combination thereof.

Contact adhesives are used in strong bonds with high shear-resistance. Pressure sensitive adhesives form a bond by the application of light pressure to bind the adhesive with the target tissue site, cannula and/or expandable member. In some embodiments, to have the device adhere to the target tissue site, pressure is applied in a direction substantially perpendicular to a surgical incision.

Multi-component adhesives harden by mixing two or more components, which chemically react. This reaction causes polymers to cross-link into acrylics, urethanes, and/or epoxies. There are several commercial combinations of multi-component adhesives in use in industry. Some of these combinations are: polyester resin-polyurethane resin; polyols-polyurethane resin, acrylic polymers-polyurethane resins or the like. The multi-component resins can be either solvent-based or solvent-less. In some embodiments, the solvents present in the adhesives are a medium for the polyester or the polyurethane resin. Then the solvent is dried during the curing process.

In some embodiments, the adhesive can be a one-part adhesive. One-part adhesives harden via a chemical reaction with an external energy source, such as radiation, heat, and moisture. Ultraviolet (UV) light curing adhesives, also known as light curing materials (LCM), have become popular within the manufacturing sector due to their rapid curing time and strong bond strength. Light curing adhesives are generally acrylic based. The adhesive can be a heat-curing adhesive, where when heat is applied (e.g., body heat), the components react and cross-link. This type of adhesive includes epoxies, urethanes, and/or polyimides. The adhesive can be a moisture curing adhesive that cures when it reacts with moisture present (e.g., bodily fluid) on the substrate surface or in the air. This type of adhesive includes cyanoacrylates or urethanes. The adhesive can have natural components, such as for example, vegetable matter, starch (dextrin), natural resins or from animals e.g. casein or animal glue. The adhesive can have synthetic components based on elastomers, thermoplastics, emulsions, and/or thermosets including epoxy, polyurethane, cyanoacrylate, or acrylic polymers.

The allograft provides a matrix for the cells to guide the process of tissue formation in vivo in three dimensions. The morphology of the allograft guides cell migration and cells are able to migrate into or over the allograft, respectively. The cells then are able to proliferate and synthesize new tissue and form bone and/or cartilage.

In some embodiments, the allograft comprises a plurality of pores. In some embodiments, at least 10% of the pores are between about 10 micrometers and about 500 micrometers at their widest points. In some embodiments, at least 20% of the pores are between about 50 micrometers and about 150 micrometers at their widest points. In some embodiments, at least 30% of the pores are between about 30 micrometers and about 70 micrometers at their widest points. In some embodiments, at least 50% of the pores are between about 10 micrometers and about 500 micrometers at their widest points. In some embodiments, at least 90% of the pores are between about 50 micrometers and about 150 micrometers at their widest points. In some embodiments, at least 95% of the pores are between about 100 micrometers and about 250 micrometers at their widest points. In some embodiments, 100% of the pores are between about 10 micrometers and about 300 micrometers at their widest points.

In some embodiments, the allograft has a porosity of at least about 30%, at least about 50%, at least about 60%, at least about 70%, at least about 90%. The pore may support ingrowth of cells, formation or remodeling of bone, cartilage and/or vascular tissue.

In some embodiments, the allograft has a density of between about 1.6 g/cm³, and about 0.05 g/cm³. In some embodiments, the allograft has a density of between about 1.1 g/cm³, and about 0.07 g/cm³. For example, the density may be less than about 1 g/cm³, less than about 0.7 g/cm³, less than about 0.6 g/cm³, less than about 0.5 g/cm³, less than about 0.4 g/cm³, less than about 0.3 g/cm³, less than about 0.2 g/cm³, or less than about 0.1 g/cm³.

The shape of the allograft may be tailored to the site at which it is to be situated. For example, it may be in the shape of a morsel, a plug, a pin, a peg, a cylinder, a block, a wedge, ring, a sheet, etc. In some embodiments, the allograft is H-shaped for placement between the spinous process.

In some embodiments, the allograft may be made by injection molding, compression molding, blow molding, thermoforming, die pressing, slip casting, electrochemical machining, laser cutting, water-jet machining, electrophoretic deposition, powder injection molding, sand casting, shell mold casting, lost tissue scaffold casting, plaster-mold casting, ceramic-mold casting, investment casting, vacuum casting, permanent-mold casting, slush casting, pressure casting, die casting, centrifugal casting, squeeze casting, rolling, forging, swaging, extrusion, shearing, spinning, powder metallurgy compaction or combinations thereof.

In some embodiments, a therapeutic agent may be disposed on or in the allograft by hand, electrospraying, ionization spraying or impregnating, vibratory dispersion (including sonication), nozzle spraying, compressed-air-assisted spraying, brushing and/or pouring. For example, a growth factor such as rhBMP-2 may be disposed on or in the allograft.

In some embodiments, the allograft may comprise sterile and/or preservative free material.

In some embodiments, the allograft can include DBM particles, and/or cells (e.g., bone, chondrogenic cells and/or tissue) seeded or attached to it.

In some embodiments, a small amount of biologic glue can be applied to attach the DBM portions to the cortical bone portion. Suitable organic glues include TISSEEL® or TISSUCOL® (fibrin based adhesive; Immuno AG, Austria), Adhesive Protein (Sigma Chemical, USA), Dow Corning Medical Adhesive B (Dow Corning, USA), fibrinogen thrombin, elastin, collagen, alginate, demineralized bone matrix, casein, albumin, keratin or the like. A composite fibrin glue can be mixed with milled cartilage from for example, a bovine fibrinogen (e.g., SIGMA F-8630), thrombin (e.g., SIGMA T-4648) and aprotinin (e.g., SIGMA A6012. Also, human derived fibrinogen, thrombin and aprotinin can be used.

Now referring to the figures, FIG. 1 illustrates a perspective view of an embodiment of a bone implant system including an allograft, such as, for example, a bone implant 12. Bone implant 12 includes a first surface 14, a second surface 16 and a third surface 22 disposed between the first and second surfaces 14, 16. First and second surfaces 14, 16 include a bioresorbable material, such as, for example, demineralized bone matrix 18. Demineralized bone matrix 18 comprises, such as, for example, demineralized bone chips. In one embodiment, demineralized bone matrix 18 comprises demineralized bone chips, fibers, powders, shards and/or the like.

In one embodiment, demineralized bone matrix 18 is disposed within a polymer mesh bag 20. In one embodiment, polymer mesh bag 20 is made of a bioresorbable material. In one embodiment, polymer mesh bag 20 is made of a non-bioresorbable material. Bag 20 maintains the demineralized bone matrix chips, fibers and/or powder in close proximity to define a substantially rectangular structure. It is contemplated that mesh bag 20 is variously shaped such that demineralized bone matrix 18 takes the form of various shapes, such as, for example, oval, oblong, triangular, square, polygonal, irregular, uniform, non-uniform, variable and/or tapered. In some embodiments, demineralized bone matrix 18 is loosely packed within mesh bag 20 such that first and second surfaces 14, 16 are pliable and can conform to certain anatomical structures in the spine.

Mesh bag 20 can be made out of any bioresorbable, non-bioresorbable and/or biocompatible natural and/or synthetic polymer. For example, mesh bag 20 may comprise poly (alpha-hydroxy acids), poly (lactide-co-glycolide) (PLGA), polylactide (PLA), polyglycolide (PG), polyethylene glycol (PEG) conjugates of poly (alpha-hydroxy acids), polyorthoesters (POE), polyaspirins, polyphosphagenes, collagen, hydrolyzed collagen, gelatin, hydrolyzed gelatin, fractions of hydrolyzed gelatin, elastin, starch, pre-gelatinized starch, hyaluronic acid, chitosan, alginate, albumin, fibrin, vitamin E analogs, such as alpha tocopheryl acetate, d-alpha tocopheryl succinate, D,L-lactide, or L-lactide, -caprolactone, dextrans, vinylpyrrolidone, polyvinyl alcohol (PVA), PVA-g-PLGA, PEGT-PBT copolymer (polyactive), methacrylates, poly (N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate), polydioxanone, methylmethacrylate (MMA), MMA and N-vinylpyyrolidone, polyamide, oxycellulose, copolymer of glycolic acid and trimethylene carbonate, polyesteramides, polyetheretherketone, polymethylmethacrylate, polyethylene terephthalate (PET), Dakron, all biocompatible fibers, stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy or combinations thereof.

In some embodiments, the biocompatible mesh bag 20 comprises a plurality of pores. In some embodiments, at least 10% of the pores are between about 10 micrometers and about 500 micrometers at their widest points. In some embodiments, at least 20% of the pores are between about 50 micrometers and about 150 micrometers at their widest points. In some embodiments, at least 30% of the pores are between about 30 micrometers and about 70 micrometers at their widest points. In some embodiments, at least 50% of the pores are between about 10 micrometers and about 500 micrometers at their widest points. In some embodiments, at least 90% of the pores are between about 50 micrometers and about 150 micrometers at their widest points. In some embodiments, at least 95% of the pores are between about 100 micrometers and about 250 micrometers at their widest points. In some embodiments, 100% of the pores are between about 10 micrometers and about 300 micrometers at their widest points.

In some embodiments, the mesh bags 20 have a porosity of at least about 30%, at least about 50%, at least about 60%, at least about 70%, at least about 90%. The pores may support ingrowth of cells, formation or remodeling of bone, cartilage and/or vascular tissue.

In some embodiments, bag 20 may comprise collagen. Exemplary collagens include human or non-human (bovine, ovine, and/or porcine), as well as recombinant collagen or combinations thereof. Examples of suitable collagen include, but are not limited to, human collagen type I, human collagen type II, human collagen type III, human collagen type IV, human collagen type V, human collagen type VI, human collagen type VII, human collagen type VIII, human collagen type IX, human collagen type X, human collagen type XI, human collagen type XII, human collagen type XIII, human collagen type XIV, human collagen type XV, human collagen type XVI, human collagen type XVII, human collagen type XVIII, human collagen type XIX, human collagen type XXI, human collagen type XXII, human collagen type XXIII, human collagen type XXIV, human collagen type XXV, human collagen type XXVI, human collagen type XXVII, and human collagen type XXVIII, or combinations thereof. Collagen further may comprise hetero- and homo-trimers of any of the above-recited collagen types. In some embodiments, the collagen comprises hetero- or homo-trimers of human collagen type I, human collagen type II, human collagen type III, or combinations thereof.

In some embodiments, bag 20 may be seeded with harvested bone cells and/or bone tissue, such as for example, cortical bone, autogenous bone, allogenic bones and/or xenogenic bone. In some embodiments, the bag 20 may be seeded with harvested cartilage cells and/or cartilage tissue (e.g., autogenous, allogenic, and/or xenogenic cartilage tissue). For example, before insertion into the target tissue site, bag 20 can be wetted with the graft bone tissue/cells, usually with bone tissue/cells aspirated from the patient, at a ratio of about 3:1, 2:1, 1:1, 1:3 or 1:2 by volume. The bone tissue/cells are permitted to soak into bag 20, and the bag 20 may be kneaded by hand, thereby obtaining a pliable consistency that may subsequently be packed into an interspinous process space.

Bag 20 may contain an inorganic material, such as an inorganic ceramic and/or bone substitute material. Exemplary inorganic materials or bone substitute materials include but are not limited to aragonite, dahlite, calcite, amorphous calcium carbonate, vaterite, weddellite, whewellite, struvite, urate, ferrihydrate, francolite, monohydrocalcite, magnetite, goethite, dentin, calcium carbonate, calcium sulfate, calcium phosphosilicate, sodium phosphate, calcium aluminate, calcium phosphate, hydroxyapatite, alpha-tricalcium phosphate, dicalcium phosphate, β-tricalcium phosphate, tetracalcium phosphate, amorphous calcium phosphate, octacalcium phosphate, BIOGLASS™, fluoroapatite, chlorapatite, magnesium-substituted tricalcium phosphate, carbonate hydroxyapatite, substituted forms of hydroxyapatite (e.g., hydroxyapatite derived from bone may be substituted with other ions such as fluoride, chloride, magnesium sodium, potassium, etc.), or combinations or derivatives thereof.

As stated above, bone implant 12 includes a third surface 22. Third surface 22 is disposed between and connected to first and second surfaces 14, 16. Third surface 22 includes a biocompatible material such that the bioresorbable material or demineralized bone matrix 18 of the first and second surfaces 14, 16 resorbs into a patient faster than the biocompatible material of third surface 22. The biocompatible material of third surface 22 can be bioresorbable or non-bioresorbable. In one embodiment, the bioresorbable, biocompatible material of the third surface 22 includes, such as, for example, cortical bone 36. Cortical bone 36 can be fully mineralized cortical bone and has the highest compressive strength of the bone implant 12. In one embodiment, first and second surfaces 14, 16 are disposed within a bioresorbable polymer mesh bag 20 while the third surface 22 is not. In another embodiment, all three surfaces 14, 16, 22 are disposed within a bioresorbable polymer mesh bag 20.

In one embodiment, third surface 22 comprises a fully resorbable material, such as, for example, PGA, PLA, collagen and/or any combination of bioresorbable polymers listed above. Third surface 22 provides structural support as first and second surfaces 14, 16 fuse with the spinal anatomy. Shortly after the first and second surfaces 14, 16 fuse with the spinal anatomy, third surface 22 fully resorbs into the patient.

In one embodiment, third surface 22 includes a non-bioresorbable material, such as, for example, stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy.

Third surface 22 extends between a first end 24 and a second end 26. First and second ends 24, 26 each include an inner surface 28 that defines a cavity 30 configured for disposal of a spinous process. Third surface 22 provides structural integrity of bone implant 12 to maintain distraction between spinous processes SP1 and SP2 so that first and second surfaces 14, 16 fuse with at least a portion of the spine, such as, for example, vertebra V1 and vertebra V2 of vertebrae V (FIG. 6). Third surface 22 is shaped similarly to the capital letter H such that cavities 30 disposed at opposing ends 24, 26 fit between adjacent spinous processes SP1 and SP2 of adjacent vertebrae V1 and V2. Other configurations are also contemplated.

Third surface 22 includes a first side 32 and a second side 34. Sides 32, 34 extend between ends 24, 26 defining a length of third surface 22 therebetween. First side 32 of third surface 22 is connected to first surface 14 and/or the mesh bag 20 that first surface 14 is contained within. Second side 34 of third surface 22 is connected to second surface 16 and/or the mesh bag 20 that second surface 16 is contained within.

It is contemplated that first and second surfaces 14, 16 are connected to third surface 22 such that first and second surfaces 14, 16 are rotatable with respect to third surface 22. Having first and second surfaces 14, 16 be rotatable with respect to third surface 22 allows first and second surfaces 14, 16 to be manipulated into a desirable position within the spine. It is further contemplated that first, second and third surfaces 14, 16, 22 can be rigidly connected such that they are substantially stationary relative to one another.

FIG. 2 illustrates a side view of bone implant 12. In one embodiment, first and second surfaces 14, 16 have a greater thickness and width than third surface 22. It is contemplated that first and second surfaces 14, 16 have various thicknesses and widths relative to third surface 22. In some embodiments, first and second surfaces 14, 16 have a non-uniform thickness and third surface 22 has a uniform thickness. It is contemplated that first, second and third surfaces 14, 16, 22 have various thicknesses, such as, for example, oval, circular, oblong, triangular, square, polygonal, irregular, uniform, non-uniform, offset, staggered, undulating, arcuate, variable and/or tapered depending on a particular application. It is contemplated that first, second and third surfaces 14, 16, 22 have a contoured cross section. In some embodiments, surfaces 14, 16 and 22 may have alternate cross section shapes, such as, for example, oval, circular, oblong, triangular, square, polygonal, irregular, uniform, non-uniform, offset, staggered, undulating, arcuate, variable and/or tapered depending on a particular application.

In one embodiment, third surface 22, like first and second surfaces 14, 16, is also disposed within polymer mesh bag 20. A fastener, such as, for example, suture 38 or zip tie is threaded through a portion of the mesh bag 20 having third surface 22 disposed therein and a portion of mesh bags 20 having first and second surfaces 14, 16 disposed therein to connect first and second surfaces 14, 16 to third surface 22.

In one embodiment, first and second surfaces 14, 16 and/or the mesh bags 20 containing the first and second surfaces 14, 16 are bonded to third surface 22 and/or the mesh bag 20 containing third surface 22 by a fastener, any adhesive described above, air drying, freeze drying, heat drying, or by using a chemical cross-linking agent.

It is envisioned that first and second surfaces 14, 16 can have mating surfaces comprising recesses and/or projections and surface 22 can have reciprocating recesses and/or projections (e.g., joints) that allow bone implant 12 to be assembled before implantation. Assembly can also include, for example, use of an adhesive material to join parts of the implant together and provide a strong interlocking fit.

In one embodiment, where the third surface 22 is not disposed in a bioresorbable polymer mesh bag 20, holes, e.g., fenestrations, can be drilled in the third surface 22 so that these holes can be used to attach first and second surfaces 14, 16 to the third surface 22. The holes are disposed substantially in a row adjacent to sides 32, 34 and extend between first and second ends 24, 26. A fastener, such as, for example, a suture 38 or zip tie is threaded through each hole and a portion of mesh bag 20 to connect first and second surfaces 14, 16 to third surface 22. In some embodiments, bone implant 12 may be joined together utilizing pins, rods, clips, or other fasteners to allow strong and easily coupling of first, second and third surfaces 14, 16, 22.

It will be understood by those of ordinary skill in the art that the demineralized bone matrix 18 of first and second surfaces 14, 16 will have lower compressive strength and more flexibility than the non-demineralized cortical bone 36 of third surface 22. In this way, the implant can be easily inserted at the target site and positioned so that the load bearing forces will be directed on the non-demineralized cortical bone 36 of bone implant 12 and the demineralized bone matrix 18 is positioned so as to reabsorb into the patient before the non-demineralized cortical bone 36. In other words, the non-demineralized cortical bone 36 of the third surface 22 is the structural support of the implant 12 that maintains attachment/positioning to the spine while the demineralized surfaces 14, 16 are reabsorbed by the patient.

In one embodiment, as shown in FIG. 3, first, second and third surfaces 14, 16, 22 define a butterfly-shaped configuration (FIG. 3). In this embodiment, first and second surfaces 14, 16 comprise demineralized bone matrix 18 in the form of densely packed bone fibers, chips, and/or powder that are adhered to one another using an adhesive or glue. It is contemplated that surfaces 14, 16 and 22 are formed from one continuous piece of cortical bone having first and surfaces 14, 16 dipped in acid to demineralized first and second surfaces 14, 16. In some embodiments, first and second surfaces 14, 16 comprise a piece of cortical bone that has been demineralized. First and second surfaces 14, 16 include a plurality of fenestrations 40 configured to receive a bone material and to increase the surface area of first and second surfaces 14, 16. It is contemplated that third surface 22 includes fenestrations 40. Fenestrations 40 are approximately 1 mm in diameter and extend through the thickness of surfaces 14, 16. Fenestrations 40 can also be configured for engagement with a bone graft instrument used for positioning bone implant 12 in an interspinous process space. The term ‘fenestrations’ includes and encompasses voids, apertures, bores, depressions, holes, indentations, grooves, channels, notches, cavities or the like.

In some embodiments, fenestrations 40 are disposed in a honeycomb configuration. In some embodiments, fenestrations 40 may be provided in any of a variety of shapes in addition to the generally circular shape shown, including but not limited to generally rectangular, oblong, curved, triangular and other polygonal or non-polygonal shapes. For example, each perforation can comprise a shape that is triangular, pyramidal, square, rectangular, pentagonal, hexagonal, heptagonal, octagonal, U-shaped, V-shaped, W-shaped, concave, crescent, or a combination thereof.

In some embodiments, fenestrations 40 comprise about less than 50% of the entire bone implant 12. In some embodiments, fenestrations 40 comprise about less than 33% of the entire bone implant 12. In some embodiments, fenestrations 40 comprise about less than 66% of the bone implant 12. In some embodiments, fenestrations 40 comprise about less than 75% of the bone implant 12.

Demineralized bone powder can be coated in or on the fenestrations 40 using a suitable adhesive, glue, binder, carrier, or in some embodiments, the demineralized bone powder can be agglomerated and packed into fenestrations 40.

In one embodiment, as shown in FIGS. 4-5, a bone implant 42, similar to bone implant 12 described above with regard to FIGS. 1-2, is provided. Bone implant 42 includes a first layer 44, similar to first and second surfaces 14, 16 described above, and a second layer 46, similar to third surface 22 described above. First layer 44 includes an upper surface 48 and a lower surface 50 attached to second layer 46. First layer 44 includes a bioresorbable material such as, for example, demineralized bone matrix 52, similar to demineralized bone matrix 18 described above. The demineralized bone matrix 52 is in the form of chips, fibers, powder and/or shards. In one embodiment, demineralized bone matrix 52 can be a single sheet of demineralized bone. Demineralized bone matrix 52 is disposed within a bioresorbable polymer mesh bag 54, similar to mesh bag 20 described above. In one embodiment, demineralized bone matrix 52 is in the form of densely packed bone fibers, chips, and/or powder that are adhered to one another.

Second layer 46 includes a long-term bioresorbable material, such as, for example, non-demineralized cortical bone attached to lower surface 50 of first layer 44. Second layer 46, like third surface 22 described above, provides structural integrity of bone implant 42 to maintain distraction between spinous processes so that first layer 44 fuses with at least a portion of the spine. First layer 44 has a width w1 defined between a first end 56 and a second end 58. Second layer 46 has a width w2 defined between a first end 60 and a second end 62 that is approximately half of width w1. Ends 56, 58 of first layer 44 are pliable such that they overhang ends 60, 62 of second layer 46, respectively. It is contemplated that first layer 44 has a greater length than second layer 44. Other configurations that achieve the same objective are also contemplated.

The bone implant 12 may also include mechanisms or features for reducing and/or preventing slippage or migration of the device during insertion. For example, one or more surfaces of the implant may include projections such as ridges or teeth (not shown) for increasing the friction between the implant and the adjacent contacting surfaces of the bone so to prevent movement of the implant after introduction to a desired location.

Growth Factors

In some embodiments, a growth factor and/or therapeutic agent may be disposed on or in the bone implant by hand, electrospraying, ionization spraying or impregnating, vibratory dispersion (including sonication), nozzle spraying, compressed-air-assisted spraying, brushing and/or pouring. For example, a growth factor such as rhBMP-2 may be disposed on or in the allograft by the surgeon before the allograft is administered or it may be available from the manufacturer beforehand.

The allograft or bone implant may comprise at least one growth factor. In one embodiment, first and second surfaces 14, 16 comprise at least one growth factor. These growth factors include osteoinductive agents (e.g., agents that cause new bone growth in an area where there was none) and/or osteoconductive agents (e.g., agents that cause in growth of cells into and/or through the allograft). Osteoinductive agents can be polypeptides or polynucleotides compositions. Polynucleotide compositions of the osteoinductive agents include, but are not limited to, isolated Bone Morphogenetic Protein (BMP), Vascular Endothelial Growth Factor (VEGF), Connective Tissue Growth Factor (CTGF), Osteoprotegerin, Growth Differentiation Factors (GDFs), Cartilage Derived Morphogenic Proteins (CDMPs), Lim Mineralization Proteins (LMPs), Platelet derived growth factor, (PDGF or rhPDGF), Insulin-like growth factor (IGF) or Transforming Growth Factor beta (TGF-beta) polynucleotides. Polynucleotide compositions of the osteoinductive agents include, but are not limited to, gene therapy vectors harboring polynucleotides encoding the osteoinductive polypeptide of interest. Gene therapy methods often utilize a polynucleotide, which codes for the osteoinductive polypeptide operatively linked or associated to a promoter or any other genetic elements necessary for the expression of the osteoinductive polypeptide by the target tissue. Such gene therapy and delivery techniques are known in the art, (See, for example, International Publication No. WO90/11092, the disclosure of which is herein incorporated by reference in its entirety). Suitable gene therapy vectors include, but are not limited to, gene therapy vectors that do not integrate into the host genome. Alternatively, suitable gene therapy vectors include, but are not limited to, gene therapy vectors that integrate into the host genome.

In some embodiments, the polynucleotide is delivered in plasmid formulations. Plasmid DNA or RNA formulations refer to polynucleotide sequences encoding osteoinductive polypeptides that are free from any delivery vehicle that acts to assist, promote or facilitate entry into the cell, including viral sequences, viral particles, liposome formulations, lipofectin, precipitating agents or the like. Optionally, gene therapy compositions can be delivered in liposome formulations and lipofectin formulations, which can be prepared by methods well known to those skilled in the art. General methods are described, for example, in U.S. Pat. Nos. 5,593,972, 5,589,466, and 5,580,859, the disclosures of which are herein incorporated by reference in their entireties.

Gene therapy vectors further comprise suitable adenoviral vectors including, but not limited to for example, those described in U.S. Pat. No. 5,652,224, which is herein incorporated by reference.

Polypeptide compositions of the isolated osteoinductive agents include, but are not limited to, isolated Bone Morphogenetic Protein (BMP), Vascular Endothelial Growth Factor (VEGF), Connective Tissue Growth Factor (CTGF), Osteoprotegerin, Growth Differentiation Factors (GDFs), Cartilage Derived Morphogenic Proteins (CDMPs), Lim Mineralization Proteins (LMPs), Platelet derived growth factor, (PDGF or rhPDGF), Insulin-like growth factor (IGF) or Transforming Growth Factor beta (TGF-beta707) polypeptides. Polypeptide compositions of the osteoinductive agents include, but are not limited to, full length proteins, fragments or variants thereof.

Variants of the isolated osteoinductive agents include, but are not limited to, polypeptide variants that are designed to increase the duration of activity of the osteoinductive agent in vivo. Preferred embodiments of variant osteoinductive agents include, but are not limited to, full length proteins or fragments thereof that are conjugated to polyethylene glycol (PEG) moieties to increase their half-life in vivo (also known as pegylation). Methods of pegylating polypeptides are well known in the art (See, e.g., U.S. Pat. No. 6,552,170 and European Pat. No. 0,401,384 as examples of methods of generating pegylated polypeptides). In some embodiments, the isolated osteoinductive agent(s) are provided as fusion proteins. In one embodiment, the osteoinductive agent(s) are available as fusion proteins with the Fc portion of human IgG. In another embodiment, the osteoinductive agent(s) are available as hetero- or homodimers or multimers. Examples of some fusion proteins include, but are not limited to, ligand fusions between mature osteoinductive polypeptides and the Fc portion of human Immunoglobulin G (IgG). Methods of making fusion proteins and constructs encoding the same are well known in the art.

Isolated osteoinductive agents are typically sterile. In a non-limiting method, sterility is readily accomplished for example by filtration through sterile filtration membranes (e.g., 0.2 micron membranes or filters). In one embodiment, the isolated osteoinductive agents include one or more members of the family of Bone Morphogenetic Proteins (“BMPs”). BMPs are a class of proteins thought to have osteoinductive or growth-promoting activities on endogenous bone tissue, or function as pro-collagen precursors. Known members of the BMP family include, but are not limited to, BMP-1, BMP-2, BMP-3, BMP-4, BMP-5, BMP-6, BMP-7, BMP-8, BMP-9, BMP-10, BMP-11, BMP-12, BMP-13, BMP-15, BMP-16, BMP-17, BMP-18 as well as polynucleotides or polypeptides thereof, as well as mature polypeptides or polynucleotides encoding the same.

BMPs utilized as osteoinductive agents comprise one or more of BMP-1; BMP-2; BMP-3; BMP-4; BMP-5; BMP-6; BMP-7; BMP-8; BMP-9; BMP-10; BMP-11; BMP-12; BMP-13; BMP-15; BMP-16; BMP-17; or BMP-18; as well as any combination of one or more of these BMPs, including full length BMPs or fragments thereof, or combinations thereof, either as polypeptides or polynucleotides encoding the polypeptide fragments of all of the recited BMPs. The isolated BMP osteoinductive agents may be administered as polynucleotides, polypeptides, full length protein or combinations thereof.

In another embodiment, isolated osteoinductive agents include osteoclastogenesis inhibitors to inhibit bone resorption of the bone tissue surrounding the site of implantation by osteoclasts. Osteoclast and osteoclastogenesis inhibitors include, but are not limited to, osteoprotegerin polynucleotides or polypeptides, as well as mature osteoprotegerin proteins, polypeptides or polynucleotides encoding the same. Osteoprotegerin is a member of the TNF-receptor superfamily and is an osteoblast-secreted decoy receptor that functions as a negative regulator of bone resorption. This protein specifically binds to its ligand, osteoprotegerin ligand (TNFSF11/OPGL), both of which are key extracellular regulators of osteoclast development.

Osteoclastogenesis inhibitors further include, but are not limited to, chemical compounds such as bisphosphonate, 5-lipoxygenase inhibitors such as those described in U.S. Pat. Nos. 5,534,524 and 6,455,541 (the contents of which are herein incorporated by reference in their entireties), heterocyclic compounds such as those described in U.S. Pat. No. 5,658,935 (herein incorporated by reference in its entirety), 2,4-dioxoimidazolidine and imidazolidine derivative compounds such as those described in U.S. Pat. Nos. 5,397,796 and 5,554,594 (the contents of which are herein incorporated by reference in their entireties), sulfonamide derivatives such as those described in U.S. Pat. No. 6,313,119 (herein incorporated by reference in its entirety), or acylguanidine compounds such as those described in U.S. Pat. No. 6,492,356 (herein incorporated by reference in its entirety).

In another embodiment, isolated osteoinductive agents include one or more members of the family of Connective Tissue Growth Factors (“CTGFs”). CTGFs are a class of proteins thought to have growth-promoting activities on connective tissues. Known members of the CTGF family include, but are not limited to, CTGF-1, CTGF-2, CTGF-4 polynucleotides or polypeptides thereof, as well as mature proteins, polypeptides or polynucleotides encoding the same.

In another embodiment, isolated osteoinductive agents include one or more members of the family of Vascular Endothelial Growth Factors (“VEGFs”). VEGFs are a class of proteins thought to have growth-promoting activities on vascular tissues. Known members of the VEGF family include, but are not limited to, VEGF-A, VEGF-B, VEGF-C, VEGF-D, VEGF-E or polynucleotides or polypeptides thereof, as well as mature VEGF-A, proteins, polypeptides or polynucleotides encoding the same.

In another embodiment, isolated osteoinductive agents include one or more members of the family of Transforming Growth Factor-beta genes (“TGFbetas”). TGF-betas are a class of proteins thought to have growth-promoting activities on a range of tissues, including connective tissues. Known members of the TGF-beta family include, but are not limited to, TGF-beta-1, TGF-beta-2, TGF-beta-3, polynucleotides or polypeptides thereof, as well as mature protein, polypeptides or polynucleotides encoding the same.

In another embodiment, isolated osteoinductive agents include one or more Growth Differentiation Factors (“GDFs”). Known GDFs include, but are not limited to, GDF-1, GDF-2, GDF-3, GDF-7, GDF-10, GDF-11, and GDF-15. For example, GDFs useful as isolated osteoinductive agents include, but are not limited to, the following GDFs: GDF-1 polynucleotides or polypeptides corresponding to GenBank Accession Numbers M62302, AAA58501, and AAB94786, as well as mature GDF-1 polypeptides or polynucleotides encoding the same. GDF-2 polynucleotides or polypeptides corresponding to GenBank Accession Numbers BC069643, BC074921, Q9UK05, AAH69643, or AAH74921, as well as mature GDF-2 polypeptides or polynucleotides encoding the same. GDF-3 polynucleotides or polypeptides corresponding to GenBank Accession Numbers AF263538, BC030959, AAF91389, AAQ89234, or Q9NR23, as well as mature GDF-3 polypeptides or polynucleotides encoding the same. GDF-7 polynucleotides or polypeptides corresponding to GenBank Accession Numbers AB158468, AF522369, AAP97720, or Q7Z4P5, as well as mature GDF-7 polypeptides or polynucleotides encoding the same. GDF-10 polynucleotides or polypeptides corresponding to GenBank Accession Numbers BC028237 or AAH28237, as well as mature GDF-10 polypeptides or polynucleotides encoding the same.

GDF-11 polynucleotides or polypeptides corresponding to GenBank Accession Numbers AF100907, NP_{—}005802 or 095390, as well as mature GDF-11 polypeptides or polynucleotides encoding the same. GDF-15 polynucleotides or polypeptides corresponding to GenBank Accession Numbers BC008962, BC000529, AAH00529, or NP004855, as well as mature GDF-15 polypeptides or polynucleotides encoding the same.

In another embodiment, isolated osteoinductive agents include Cartilage Derived Morphogenic Protein (CDMP) and Lim Mineralization Protein (LMP) polynucleotides or polypeptides. Known CDMPs and LMPs include, but are not limited to, CDMP-1, CDMP-2, LMP-1, LMP-2, or LMP-3.

CDMPs and LMPs useful as isolated osteoinductive agents include, but are not limited to, the following CDMPs and LMPs: CDMP-1 polynucleotides and polypeptides corresponding to GenBank Accession Numbers NM_{—}000557, U13660, NP_{—}000548 or P43026, as well as mature CDMP-1 polypeptides or polynucleotides encoding the same. CDMP-2 polypeptides corresponding to GenBank Accession Numbers or P55106, as well as mature CDMP-2 polypeptides. LMP-1 polynucleotides or polypeptides corresponding to GenBank Accession Numbers AF345904 or AAK30567, as well as mature LMP-1 polypeptides or polynucleotides encoding the same. LMP-2 polynucleotides or polypeptides corresponding to GenBank Accession Numbers AF345905 or AAK30568, as well as mature LMP-2 polypeptides or polynucleotides encoding the same. LMP-3 polynucleotides or polypeptides corresponding to GenBank Accession Numbers AF345906 or AAK30569, as well as mature LMP-3 polypeptides or polynucleotides encoding the same.

In another embodiment, isolated osteoinductive agents include one or more members of any one of the families of Bone Morphogenetic Proteins (BMPs), Connective Tissue Growth Factors (CTGFs), Vascular Endothelial Growth Factors (VEGFs), Osteoprotegerin or any of the other osteoclastogenesis inhibitors, Growth Differentiation Factors (GDFs), Cartilage Derived Morphogenic Proteins (CDMPs), Lim Mineralization Proteins (LMPs), or Transforming Growth Factor-betas (TGF-betas), bone marrow aspirate, concentrated bone marrow aspirate, TP508 (an angiogenic tissue repair peptide), as well as mixtures or combinations thereof.

In some embodiments, first and second surfaces 14, 16 include mesenchymal cells, antibiotics, anti-infective compositions and combinations thereof.

In another embodiment, the one or more isolated osteoinductive agents useful in the bioactive formulation are selected from the group consisting of BMP-1, BMP-2, BMP-3, BMP-4, BMP-5, BMP-6, BMP-7, BMP-8, BMP-9, BMP-10, BMP-11, BMP-12, BMP-13, BMP-15, BMP-16, BMP-17, BMP-18, or any combination thereof; CTGF-1, CTGF-2, CGTF-3, CTGF-4, or any combination thereof; VEGF-A, VEGF-B, VEGF-C, VEGF-D, VEGF-E, or any combination thereof; GDF-1, GDF-2, GDF-3, GDF-7, GDF-10, GDF-11, GDF-15, or any combination thereof; CDMP-1, CDMP-2, LMP-1, LMP-2, LMP-3, and or combination thereof; Osteoprotegerin; TGF-beta-1, TGF-beta-2, TGF-beta-3, or any combination thereof; or any combination of one or more members of these groups.

The concentrations of growth factor can be varied based on the desired length or degree of osteogenic effects desired. Similarly, one of skill in the art will understand that the duration of sustained release of the growth factor can be modified by the manipulation of the compositions comprising the sustained release formulation, such as for example, modifying the percent of allograft found within a sustained release formulation, microencapsulation of the formulation within polymers, including polymers having varying degradation times and characteristics, and layering the formulation in varying thicknesses in one or more degradable polymers. These sustained release formulations can therefore be designed to provide customized time release of growth factors that simulate the natural healing process.

In some embodiments, a statin may be used as the growth factor. Statins include, but is not limited to, atorvastatin, simvastatin, pravastatin, cerivastatin, mevastatin (see U.S. Pat. No. 3,883,140, the entire disclosure is herein incorporated by reference), velostatin (also called synvinolin; see U.S. Pat. Nos. 4,448,784 and 4,450,171 these entire disclosures are herein incorporated by reference), fluvastatin, lovastatin, rosuvastatin and fluindostatin (Sandoz XU-62-320), dalvastain (EP Appln. Publn. No. 738510 A2, the entire disclosure is herein incorporated by reference), eptastatin, pitavastatin, or pharmaceutically acceptable salts thereof or a combination thereof. In various embodiments, the statin may comprise mixtures of (+)R and (−)-S enantiomers of the statin. In various embodiments, the statin may comprise a 1:1 racemic mixture of the statin.

The growth factor may contain inactive materials such as buffering agents and pH adjusting agents such as potassium bicarbonate, potassium carbonate, potassium hydroxide, sodium acetate, sodium borate, sodium bicarbonate, sodium carbonate, sodium hydroxide or sodium phosphate; degradation/release modifiers; drug release adjusting agents; emulsifiers; preservatives such as benzalkonium chloride, chlorobutanol, phenylmercuric acetate and phenylmercuric nitrate, sodium bisulfate, sodium bisulfite, sodium thiosulfate, thimerosal, methylparaben, polyvinyl alcohol and phenylethyl alcohol; solubility adjusting agents; stabilizers; and/or cohesion modifiers. In some embodiments, the growth factor may comprise sterile and/or preservative free material.

These above inactive ingredients may have multi-functional purposes including the carrying, stabilizing and controlling the release of the growth factor and/or other therapeutic agent(s). The sustained release process, for example, may be by a solution-diffusion mechanism or it may be governed by an erosion-sustained process.

In some embodiments, the growth factor is supplied in an aqueous buffered solution. Exemplary aqueous buffered solutions include, but are not limited to, TE, HEPES (2-[4-(2-hydroxyethyl)-1-piperazinyl]ethanesulfonic acid), MES (2-morpholinoethanesulfonic acid), sodium acetate buffer, sodium citrate buffer, sodium phosphate buffer, a Tris buffer (e.g., Tris-HCL), phosphate buffered saline (PBS), sodium phosphate, potassium phosphate, sodium chloride, potassium chloride, glycerol, calcium chloride or a combination thereof. In various embodiments, the buffer concentration can be from about 1 mM to 100 mM.

In some embodiments, the BMP-2 is provided in a vehicle (including a buffer) containing sucrose, glycine, L-glutamic acid, sodium chloride, and/or polysorbate 80.

Additional Therapeutic Agents

The growth factors of the present application may be disposed on or in the bone implant with other therapeutic agents. For example, the growth factor may be disposed on or in the bone implant by electrospraying, ionization spraying or impregnating, vibratory dispersion (including sonication), nozzle spraying, compressed-air-assisted spraying, brushing and/or pouring.

Exemplary therapeutic agents include but are not limited to IL-1 inhibitors, such Kineret® (anakinra), which is a recombinant, non-glycosylated form of the human inerleukin-1 receptor antagonist (IL-1Ra), or AMG 108, which is a monoclonal antibody that blocks the action of IL-1. Therapeutic agents also include excitatory amino acids such as glutamate and aspartate, antagonists or inhibitors of glutamate binding to NMDA receptors, AMPA receptors, and/or kainate receptors. Interleukin-1 receptor antagonists, thalidomide (a TNF-α release inhibitor), thalidomide analogues (which reduce TNF-α production by macrophages), quinapril (an inhibitor of angiotensin II, which upregulates TNF-α), interferons such as IL-11 (which modulate TNF-α receptor expression), and aurin-tricarboxylic acid (which inhibits TNF-α), may also be useful as therapeutic agents for reducing inflammation. It is further contemplated that where desirable a pegylated form of the above may be used. Examples of still other therapeutic agents include NF kappa B inhibitors such as antioxidants, such as dilhiocarbamate, and other compounds, such as, for example, sulfasalazine.

Examples of therapeutic agents suitable for use also include, but are not limited to an anti-inflammatory agent, analgesic agent, or osteoinductive growth factor or a combination thereof. Anti-inflammatory agents include, but are not limited to, apazone, celecoxib, diclofenac, diflunisal, enolic acids (piroxicam, meloxicam), etodolac, fenamates (mefenamic acid, meclofenamic acid), gold, ibuprofen, indomethacin, ketoprofen, ketorolac, nabumetone, naproxen, nimesulide, salicylates, sulfasalazine[2-hydroxy-5-[-4-[C2-pyridinylamino)sulfonyl]azo]benzoic acid, sulindac, tepoxalin, and tolmetin; as well as antioxidants, such as dithiocarbamate, steroids, such as cortisol, cortisone, hydrocortisone, fludrocortisone, prednisone, prednisolone, methylprednisolone, triamcinolone, betamethasone, dexamethasone, beclomethasone, fluticasone or a combination thereof.

Suitable analgesic agents include, but are not limited to, acetaminophen, bupivicaine, fluocinolone, lidocaine, opioid analgesics such as buprenorphine, butorphanol, dextromoramide, dezocine, dextropropoxyphene, diamorphine, fentanyl, alfentanil, sufentanil, hydrocodone, hydromorphone, ketobemidone, levomethadyl, mepiridine, methadone, morphine, nalbuphine, opium, oxycodone, papaveretum, pentazocine, pethidine, phenoperidine, piritramide, dextropropoxyphene, remifentanil, tilidine, tramadol, codeine, dihydrocodeine, meptazinol, dezocine, eptazocine, flupirtine, amitriptyline, carbamazepine, gabapentin, pregabalin, or a combination thereof.

In various embodiments, a kit is provided that may include additional parts along with the bone implant to be used to implant the bone implant. The kit may include the bone implant in a first compartment. The second compartment may include the growth factor and any other instruments needed for implanting the bone implant. A third compartment may include gloves, drapes, wound dressings and other procedural supplies for maintaining sterility during the implanting process, as well as an instruction booklet. A fourth compartment may include additional tools for implantation (e.g., drills, drill bits, bores, punches, etc.). Each tool may be separately packaged in a plastic pouch that is radiation sterilized. A fifth compartment may comprise an agent for radiographic imaging or the agent may be disposed on the allograft and/or carrier to monitor placement and tissue growth. A cover of the kit may include illustrations of the implanting procedure and a clear plastic cover may be placed over the compartments to maintain sterility.

It will be apparent to those skilled in the art that various modifications and variations can be made to various embodiments described herein without departing from the spirit or scope of the teachings herein. Thus, it is intended that various embodiments cover other modifications and variations of various embodiments within the scope of the present teachings.

What is claimed is:

1. A bone implant, comprising:
a first surface and a second surface, the first and second surfaces comprising a bioresorbable material;
a third surface comprising a biocompatible material disposed between and connected to the first and second surfaces, the third surface extending between a first end and a second end, the first and second ends each including an inner surface defining a cavity configured for disposal of a spinous process;
wherein the bioresorbable material of the first and second surfaces is a faster resorbing material than the biocompatible material of the third surface; and
wherein the third surface provides structural integrity of the implant to maintain distraction between spinous processes so that the first and second surfaces fuse with at least a portion of the spine.

2. The bone implant as recited in claim 1, wherein the bioresorbable material of the first and second surfaces comprises demineralized bone matrix and the biocompatible material of the third surface comprises a bioresorbable material.

3. The bone implant as recited in claim 2, wherein the bioresorbable material comprises non-demineralized cortical bone.

4. The bone implant as recited in claim 2, wherein the demineralized bone matrix includes demineralized bone chips.

5. The bone implant as recited in claim 1, wherein the bioresorbable material of the first and second surfaces comprises demineralized bone matrix and the biocompatible material of the third surface comprises a non-bioresorbable material, the non-bioresorbable material comprising at least one of stainless steel alloys, commercially pure titanium, titanium alloys, Grade 5 titanium, cobalt-chrome alloys, stainless steel alloys, calcium phosphate, polyaryletherketone (PAEK), polyetheretherketone (PEEK), polyetherketoneketone (PEKK), polyetherketone (PEK), carbon-PEEK composites and PEEK-BaSO₄.

6. The bone implant as recited in claim 2, wherein the first and second surfaces are each disposed within a biocompatible polymer mesh bag.

7. The bone implant as recited in claim 6, wherein the polymer mesh bag is bioresorbable.

8. The bone implant as recited in claim 6, wherein the mesh bags of the first and second surfaces are bonded to the third surface by a fastener, an adhesive, air drying, freeze drying, heat drying, or by using a chemical cross-linking agent and/or interlocking parts.

9. The bone implant as recited in claim 6, wherein the third surface is disposed within a biocompatible polymer mesh bag, the mesh bags of the first and second surfaces being attached to the mesh bag of the third surface.

10. The bone implant as recited in claim 9, wherein the biocompatible polymer mesh bag of the third surface is bioresorbable.

11. The bone implant as recited in claim 2, wherein the first, second and third surfaces define a butterfly shape and are formed from one continuous piece of cortical bone.

12. The bone implant as recited in claim 11, wherein the first and second surfaces comprise a plurality of fenestrations configured to receive a bone material, to increase flexibility and/or to increase the surface area of the first and second surfaces.

13. A bone implant, comprising:
a first layer including an upper surface and a lower surface, the first layer comprising a bioresorbable material;
a second layer comprising a biocompatible material attached to the lower surface of the first layer, the second layer extending between a first end and a second end, the first and second ends each including an inner surface defining a cavity configured for disposal of a spinous process;
wherein the bioresorbable material of the first layer is a faster resorbing material than the biocompatible material of the second layer; and
wherein the second layer provides structural integrity of the implant to maintain distraction between spinous processes so that the first layer fuses with at least a portion of the spine.

14. The bone implant as recited in claim 13, wherein the bioresorbable material of the first layer comprises demineralized bone matrix and the biocompatible material of the second layer comprises non-demineralized cortical bone.

15. The bone implant as recited in claim 13, wherein the bioresorbable material of the first layer comprises demineralized bone matrix and the biocompatible material of the second layer comprises a non-bioresorbable material, the non-bioresorable material comprising at least one of stainless steel alloys, commercially pure titanium, titanium alloys, Grade 5 titanium, cobalt-chrome alloys, stainless steel alloys, calcium phosphate, polyaryletherketone (PAEK), polyetheretherketone (PEEK), polyetherketoneketone (PEKK), polyetherketone (PEK), carbon-PEEK composites and PEEK-BaSO₄.

16. The bone implant as recited in claim 14, wherein the first layer is disposed within a biocompatible polymer mesh bag.

17. The bone implant as recited in claim 16, wherein the polymer mesh bag is bioresorbable.

18. The bone implant as recited in claim 14, wherein the demineralized bone matrix includes demineralized bone chips.

19. A bone implant, comprising:
a first bioresorbable polymer mesh bag and a second bioresorbable polymer mesh bag, the first and second mesh bags each comprising demineralized bone chips disposed therein; and
a surface comprising cortical bone, the surface being disposed between and connected to the first and second mesh bags, the surface extending between a first end and a second end, the first and second ends each including an inner surface defining a cavity configured for disposal of a spinous process,
wherein the surface provides structural integrity of the implant to maintain distraction between spinous processes so that the demineralized bone chips fuse with at least a portion of the spine.

20. A bone implant as recited in claim 19, wherein the surface is disposed in a third bioresorbable polymer mesh bag, the first and second mesh bags being attached to the third mesh bag.
